(19) United States
(12) Patent Application Publication
(10) Pub. No.: US 2023/0010633 A1
(43) Pub. Date: Jan. 12, 2023

(54) DNA LOCATION METHOD AND APPARATUS

(71) Applicant: Peter J. Wilk, New York, NY (US)

(72) Inventor: Peter J. Wilk, New York, NY (US)

(21) Appl. No.: 17/861,218

(22) Filed: Jul. 10, 2022

Related U.S. Application Data
(60) Provisional application No. 63/220,482, filed on Jul. 10, 2021.

(51) Int. Cl. C12Q 1/6825

(52) U.S. Cl. CPC C12Q 1/6825

(57) ABSTRACT

A detector, and method, for detecting nucleic acids includes a transmitter adapted to transmit light at 260 nm over an area of illumination, a receiver adapted to receive light at 260 nm from the area of illumination, a comparator adapted to compare the amplitude of light received by the receiver to a background level and to produce a hot-spot signal indicative of the presence of a nucleic acid when the amplitude is attenuated relative to the background level, and a display adapted to display the hot-spot. Preferably, the detector further includes a photographic detector adapted to receive an optical background image over an area including the area of illumination, the display adapted to display the optical background image and the hot-spot within the optical background image.

### CROSS REFERENCE TO RELATED APPLICATION

The present invention claims the benefit of provisional U.S. application Ser. No. 63/220,482, filed Jul. 10, 2021, the entire disclosure of which is hereby incorporated by reference.

### FIELD OF THE INVENTION

The present invention is directed to a novel technique for detecting and locating DNA in situ.

### BACKGROUND OF THE INVENTION

Since the invention of the polymerase chain reaction, DNA analysis has become one of the most valuable tools for a multitude of applications in scientific research. Such analysis naturally requires a sample of DNA for testing.

The nucleic acids DNA and RNA strongly absorb UV light at 260 nm, producing photochemical reactions in the nucleic acids. This phenomenon has been used to detect the presence and quality of DNA and RNA in a given sample, in a laboratory setting. For example, the Photopette® Bio UV-Vis Spectrophotometer made by Tip Biosystems can be used to determine DNA concentration and purity measurements of a sample. This and similar techniques, however, require a “trial and error” laboratory analysis of preselected samples which may or may not include DNA and/or RNA. As such, these techniques and instruments are not designed for locating nucleic acids in the environment, in situ, as opposed to the testing of preselected samples. Yet, the ability to locate potential sources of DNA/RNA in situ, such as an archeological dig or crime scene, would be extremely valuable.

### SUMMARY OF THE INVENTION

Accordingly, the present invention is directed to apparatuses and methods for finding and locating nucleic acids in the environment, in situ. Since nucleic acids, such as DNA and RNA, strongly absorb UV light at wavelengths of 260 nm, the absorbed energy inducing photochemical reactions in the nucleic acid, the present invention detects this absorption in 260 nm light reflected by the nucleic acid, to determine the presence of the nucleic acid in situ. As will be discussed, the present invention first establishes a baseline reflectivity at 260 nm, then transmits and receives UV at 260 nm over a field of interest, compares the reflected UV to the baseline, and detects a DNA “hotspot” when the received UV reflected from the field of interest is attenuated relative to the baseline. The hotspot is superimposed on the optical image of the field of interest to locate a potential source of DNA.

In accordance with one aspect of the invention, a detector for detecting nucleic acids comprises a transmitter adapted to transmit light at 260 nm over an area of illumination, a receiver adapted to receive light at 260 nm from the area of illumination, a comparator adapted to compare the amplitude of light received by the receiver to a background level and to produce a hot-spot signal indicative of the presence of a nucleic acid when the amplitude is attenuated relative to the background level, and a display adapted to display the hot-spot. Preferably, the detector further includes a photographic detector adapted to receive an optical background image over an area including the area of illumination, the display adapted to display the optical background image and the hot-spot within the optical background image.

The detector may further include a memory adapted to store (i) the optical background image, (ii) the hot-spot within the optical background image, and (iii) GPS coordinates of the background image. Preferably, the transmitter, the receiver, the comparator and the display are contained in a single unit. Further, the light transmitted by the transmitter can be collimated, and the background level can be established by scanning the transmitter and receiver over an area exceeding the desired resolution of the detection unit and averaging the amplitudes received by the receiver. Additionally, the detector can include a connection to at least one of a smartphone, tablet and computer.

In accordance with another aspect of the invention, a method for detecting nucleic acids includes transmitting light at 260 nm over an area of illumination, receiving light at 260 nm from the area of illumination, comparing the amplitude of light received by the receiver to a background level, and producing a hot-spot signal indicative of the presence of a nucleic acid when the amplitude is attenuated relative to the background level, and displaying the hot-spot. Preferably, the method further includes receiving from a photographic detector an optical background image over an area including the area of illumination, and displaying the optical background image and the hot-spot within the optical background image. The method may further include storing in a memory (i) the optical background image, (ii) the hot-spot within the optical background image, and (iii) GPS coordinates of the background image. If desired, the method may include collimating the light transmitted by the transmitter. The background level may be determined by scanning the transmitter and receiver over an area exceeding the desired resolution of the detection unit and averaging the amplitudes received by the receiver. Finally, the method may further include transmitting (i) the optical background image, (ii) the hot-spot within the optical background image, and (iii) GPS coordinates of the background image to at least one of a smartphone, tablet and computer.

### BRIEF DESCRIPTION OF THE DRAWINGS

These and other objects and aspects of the present invention will be described with reference to the following drawing figures, of which:

FIG. 1 is an illustration of the absorption spectrum of DNA and RNA showing strong absorption at 260 nm;

FIG. 2 is an illustration of an example of apparatus for detecting nucleic acids in situ in accordance with one aspect of the present invention;

FIG. 3 is an illustration of an exemplary method employed by the apparatus of FIG. 2 for detecting nucleic acids in the environment; and

FIG. 4 is an illustration of an example of a system, embodied in the apparatus of FIG. 2, for implementing the apparatus and method of the present invention.

### DETAILED DESCRIPTION

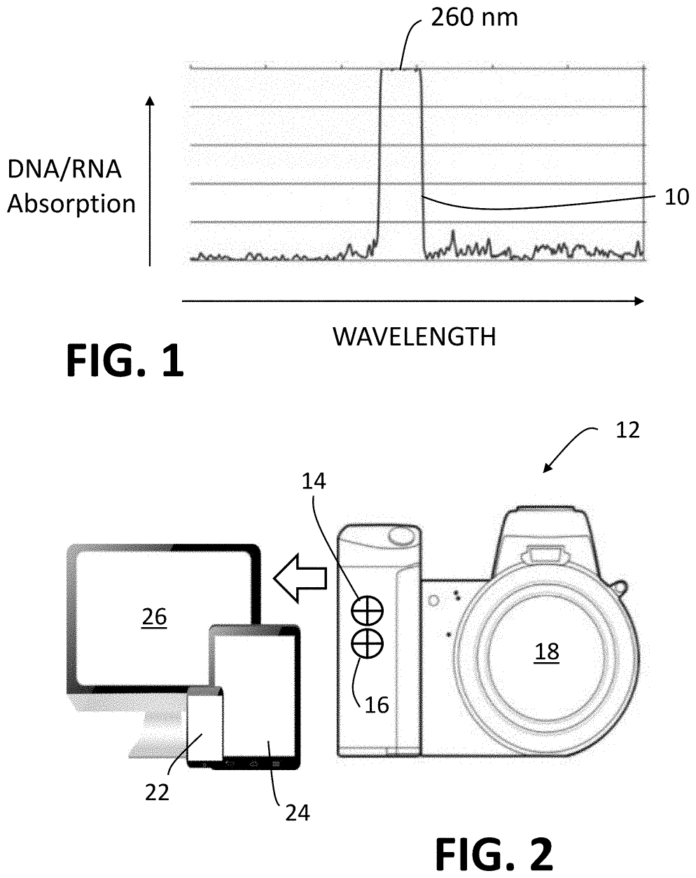

FIG. 1 illustrates the absorption spectrum of DNA and RNA. It is well-known that nucleic acids, such as DNA and RNA, strongly absorb UV light at wavelengths of 260 nm. This is shown in the figure, where the peak 10 in the absorption curve is shown centered around 260 nm. The energy absorbed by the nucleic acid at 260 nm induces photochemical reactions in the nucleic acid. The present invention takes advantage of this fact, and the concomitant reduction in 260 nm light reflected by the nucleic acid, to detect the presence of the nucleic acid in situ.

An example of apparatus for detecting nucleic acids in situ in accordance with one aspect of the present invention is shown in FIG. 2. A portable detection unit 12 comprises a UV transmitter 14 and a co-located UV receiver 16, which are designed to transmit and receive 260 nm light, respectively, over an area of illumination. The transmitter 14 may produce a collimated beam of light, and is preferably comprised of a laser diode, transmitting at approximately 260 nm. Preferably, the beam is collimated to an area having a size that is a function of the desired resolution of the detection unit. Alternatively, the area illuminated by the transmitter can be larger than the desired resolution of the detection unit, as long as the receiver, described below, resolves the detected signal at the desired resolution of the detection unit.

The receiver 16 is preferably adapted to detect an area substantially equal or similar in size to the area of the desired resolution of the detection unit, which may be equal in area to a collimated beam produced by transmitter 14, if a collimated beam is used. Moreover, the transmitter and receiver are collocated, and co-directed, such that they transmit to, and receive from, the same area in space. As an alternative, the receiver 16 can be incorporated into a photographic detector 18, described below, as long as the transmitter and receiver transmit to, and receive from, same area in space.

The transmitter 14 and receiver 16 can be scanned, in unison, over a desired field of view either electronically, by beam-steering circuitry in a well-known manner, or manually by a user by physically moving the detection unit back and forth over the desired field of view.

As noted, the detection unit 12 also includes a photographic detector 18, which includes the functionality of an electronic camera. In addition, the photo detector 18 is adapted to detect an area at least great as the desired resolution of the detection unit, and is electronically steered in sync with the transmitter 14 and receiver 16 when electronic steering is employed by the transmitter 14 and receiver 16; however, such steering of the photo detector 18 is not required if the transmitter 14 and receiver 16 are scanned manually, as the entire detection unit 12, including the photodetector 18, will move along with the transmitter 14 and receiver 16.

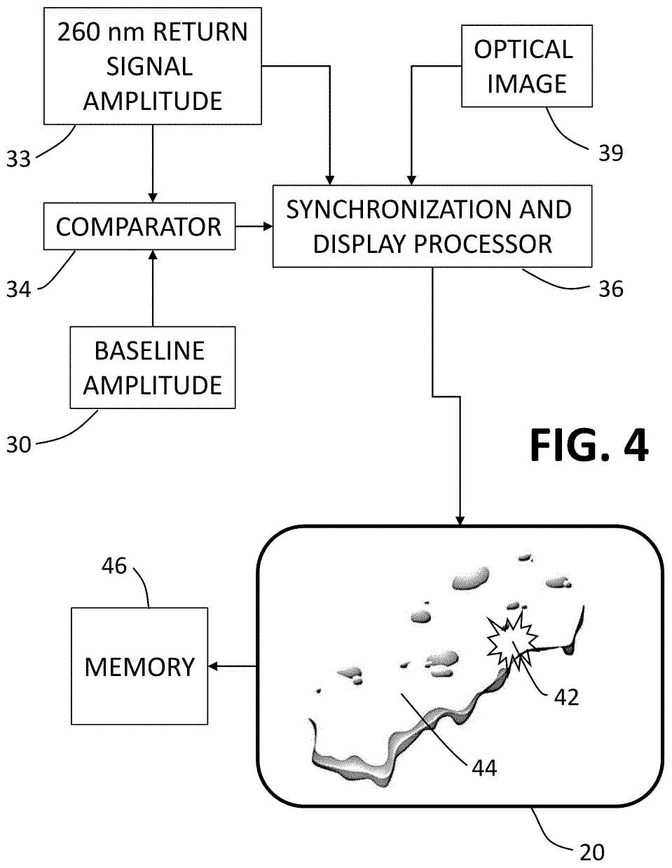

The opposite side of the detection unit 12 (not shown in FIG. 2) includes a display 20, FIG. 4, in a manner similar to a standard electronic camera. In addition, the detection unit can be connected by hardwire, or through a wireless connection, to one or more of co-located or remote display units such as smart phone 22, tablet 24 or computer 26, which are adapted to display at least the same information provided by photo display 20, to potentially remote users.

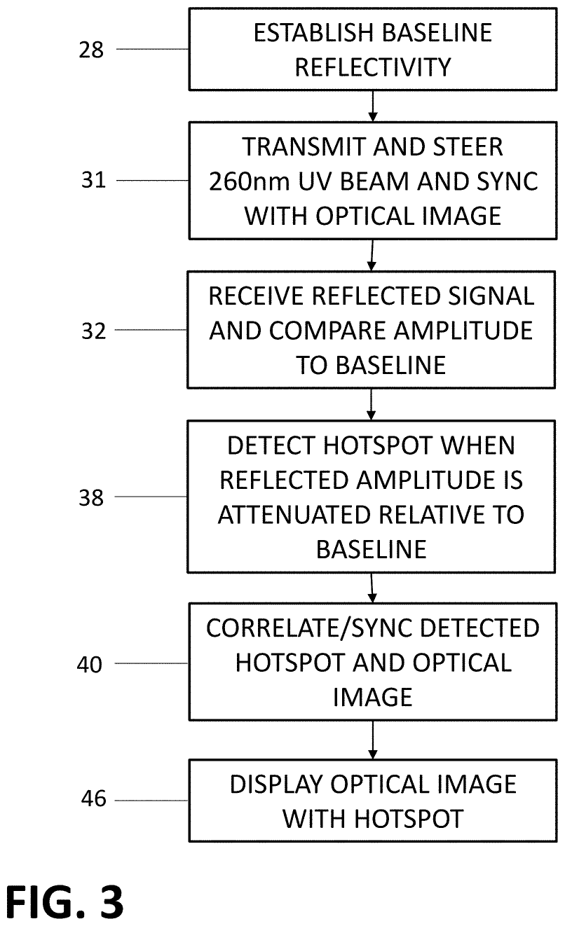

With reference to FIGS. 3 and 4, in operation, a user first must establish a baseline of reflectivity of 260 nm UV light over a desired field of view, as shown at block 28. This entails scanning, electronically or manually, over a large field of view, encompassing at least all of the field of view of interest, and averaging the return attenuation at 260 nm. Reasonably assuming that the entire field of view is not covered with nucleic acid, the average will establish a baseline for the detection of nucleic acid. The field of view should be chosen to be at least somewhat homogeneous in terms of background optics, so that large variations in reflectivity, not due to absorption by nucleic acids, does not occur (which might happen if, for example, both grassy terrain and a body of water are included in a single field of view). The baseline or average 260 nm reflectivity amplitude is stored in a memory location 30, FIG. 4, which resides in detection unit 12.

After determining the baseline reflectivity, the transmitter 14 and detector 16 are steered, in unison, over the desired field of view, at block 31, FIG. 3, the transmitter transmitting a beam of UV at 260 nm, and the detector receiving the reflected signal at block 32. The amplitude of the reflected signal, represented by block 33, FIG. 4, is compared to the baseline in comparator 34, FIG. 4, in the detection unit 12. If the reflected signal is significantly attenuated, by more than a preselected value, relative to the baseline, the synchronization and display processor 36, FIG. 4, in the detection unit will detect a “hotspot” at block 38, FIG. 3. The synchronization and display processor 36, which also receives the optical image signal as shown in FIG. 4 at input 39, correlates and syncs the detected hotspot and the optical image at block 40, FIG. 3. The display processor 36 then displays the hot spot 42 on the display 20, FIG. 4, superimposed on the optical image 44, in the desired field of view, at block 46, FIG. 3. Depending on the type of scanning employed, the optical image can be “stitched” in a well-known manner to generate the full view of the entire field of view. Further, the comparator can compare different levels of attenuation and assign different color codes for the hotspot depending on the attenuation level, highlighting specific hotspots that are greatly attenuated, which would be indicative of a DNA-rich spot.

Thus, the hotspot 42 can be seen in the field of view on the display 20, superimposed on the background 44 for reference. At this point the user can retrieve samples from the hotspot with a good level of confidence that DNA or other nucleic acid will be found. The superimposed image can be stored in memory 46, FIG. 4, in the detection unit, along with GPS coordinates for the image so that samples may be taken from the hotspot at a later time. Such information is also preferably transmitted to one or more of the phone 22, tablet 24 and computer 26, for storage and distribution to others having access to such information.

The examples disclosed herein are for exemplary purposes only and should not be construed as limiting the present invention, which is defined in the following claims.

I claim:

1. Apparatus for detecting nucleic acids comprising:
a transmitter adapted to transmit light at 260 nm over an area of illumination;
a receiver adapted to receive light at 260 nm from said area of illumination;
a comparator adapted to compare the amplitude of light received by said receiver to a background level, and to produce a hot-spot signal indicative of the presence of a nucleic acid when said amplitude is attenuated relative to said background level; and
a display adapted to display said hot-spot.

2. The apparatus of claim 1, further comprising a photographic detector adapted to receive an optical background image over an area including said area of illumination, said display adapted to display said optical background image and said hot-spot within said optical background image.

3. The apparatus of claim 2 further comprising a memory adapted to store (i) said optical background image, (ii) said hot-spot within said optical background image, and (iii) GPS coordinates of said background image.

4. The apparatus of claim 2 wherein said transmitter, said receiver, said comparator and said display are contained in a single unit.

5. The apparatus of claim 2 wherein said light transmitted by said transmitter is collimated.

6. The apparatus of claim 2 wherein said background level is established by scanning said transmitter and receiver over an area exceeding the desired resolution of the detection unit and averaging the amplitudes received by said receiver.

7. The apparatus of claim 3 wherein said apparatus further comprises a connection to at least one of a smartphone, tablet and computer.

8. A method for detecting nucleic acids comprising:
transmitting light at 260 nm over an area of illumination;
receiving light at 260 nm from said area of illumination;
comparing the amplitude of light received by said receiver to a background level, and producing a hot-spot signal indicative of the presence of a nucleic acid when said amplitude is attenuated relative to said background level; and
displaying said hot-spot.

9. The method of claim 8, further comprising receiving from a photographic detector an optical background image over an area including said area of illumination, and displaying said optical background image and said hot-spot within said optical background image.

10. The method of claim 9 further comprising storing in a memory (i) said optical background image, (ii) said hot-spot within said optical background image, and (iii) GPS coordinates of said background image.

11. The method of claim 9 wherein said light transmitted by said transmitter is collimated.

12. The method of claim 9 further comprising establishing said background level by scanning said transmitter and receiver over an area exceeding the desired resolution of the detection unit and averaging the amplitudes received by said receiver.

13. The method of claim 10 further comprising transmitting (i) said optical background image, (ii) said hot-spot within said optical background image, and (iii) GPS coordinates of said background image to at least one of a smartphone, tablet and computer.
